(19) United States
(12) Patent Application Publication
(10) Pub. No.: US 2013/0345678 A1
(43) Pub. Date: Dec. 26, 2013

(54) TREATMENT OF EXCESSIVE MENSTRUAL BLOOD LOSS BY FEMININE SANITARY PRODUCTS MEDICATED WITH ANTIFIBRINOLYTIC OR HEMOSTATIC AGENT

(71) Applicant: ARSTAT Inc., Flemington, NJ (US)

(72) Inventor: Arkady RUBIN, Flemington, NJ (US)

(73) Assignee: ARSTAT Inc., Flemington, NJ (US)

(21) Appl. No.: 13/973,337

(22) Filed: Aug. 22, 2013

Related U.S. Application Data
(63) Continuation of application No. PCT/US2011/061684, filed on Nov. 21, 2011.
(60) Provisional application No. 61/448,385, filed on Mar. 2, 2011.

(51) Int. Cl. A61M 31/00

(52) U.S. Cl. CPC A61M 31/002
USPC 604/515; 604/286; 514/561; 514/13.7; 514/538; 424/430; 424/431

(57) ABSTRACT

A method, medicated feminine sanitary products and compositions for reducing menstrual blood loss in a female are provided. The method comprises delivering an active agent to the uterine cavity by administering intravaginally to the female a therapeutically effective amount of the active agent on a medicated feminine sanitary product during menstrual bleeding, wherein the medicated feminine sanitary product is routinely utilized to absorb the menstrual flow, wherein the active agent is incorporated in the medicated feminine sanitary product as a pharmaceutically acceptable composition, the active agent is any of an antifibrinolytic and a hemostatic agent.

### CROSS-REFERENCE TO RELATED APPLICATIONS

This application is a continuation of international application PCT/US2011/061684 filed Nov. 21, 2011, which claims the benefit of U.S. provisional patent application No. 61/448,385 filed Mar. 2, 2011, the contents of which are incorporated by reference.

### TECHNICAL FIELD

The present invention relates to the treatment of female menstrual disorders and control of the amount of menstrual blood loss. More specifically, the present invention relates to the reduction of menstrual blood loss achievable by intravaginal delivery of an antifibrinolytic or hemostatic agent. More specifically, the present invention relates to the intravaginal delivery of an antifibrinolytic or hemostatic agent using medicated feminine sanitary products (e.g., menstrual pads, sanitary napkins, sanitary towels, sanitary pads, or menstrual tampons).

### BACKGROUND

There are tens of millions of menstruating women in the United States. A woman's menstrual bleeding pattern plays a significant role in her life, influencing her work productivity, her social and leisure activities, as well as her physical and psychological state of well-being.

If a woman's periods are so heavy or so long that she finds them distressing she is experiencing heavy menstrual bleeding. A volume of menstrual blood loss (MBL) exceeding 80 mL per menstrual cycle is defined as menorrhagia. One-third of all women experience heavy menstrual bleeding at some point in their lives, and in Western countries about 5% of reproductive-aged women seek treatment for it annually.¹

A woman's self-assessment of the intensity of menstrual bleeding is always subjective. One half of women who report heavy menstrual bleeding have menstrual blood loss (MBL)<80 mL. One third of women who report moderate or light cycles have MBL>80 mL.² Oftentimes, irrespective of the amount of MBL, a woman with complaints about heavy menstrual periods visits her physician and requests medical assistance. Appropriate therapy, including pharmacological treatments, may then be initiated.

Moderate menstrual periods may also have a negative impact on a woman's quality of life. Days of relatively heavy bleeding and/or a prolonged duration of menstrual periods contribute to physical and psychological discomfort as well as to interference with social and leisure activities. Even episodes of the lightest bleeding (“spotting”) may impact a woman's well-being and daily routines.

Women with heavy menstrual bleeding are frequently offered off-label use of approved hormonal contraceptives. Due to known safety issues, danazol is rarely considered as a viable pharmacological treatment option. NSAIDs are also used for the treatment of women suffering from excessive menstrual blood loss, particularly when the menstrual periods are painful and/or a woman is clinically diagnosed with dysmenorrhea. Surgical procedures (generally hysterectomy) may be considered for women with severe menorrhagia. Yet, removal of the uterus is a radical treatment option with known undesirable consequences, including loss of fertility and surgical morbidity, as well as entailing high cost.

Among non-hormonal medications, oral tranexamic acid is considered as a first-line treatment option for the treatment of menorrhagia.⁴ Oral tranexamic acid is marketed in the US as Lysteda® and both within and outside the US as Cyklokapron®. As is reported on the Lysteda label, tranexamic acid is a synthetic lysine amino acid derivative, which diminishes the dissolution of hemostatic fibrin by plasmin. In the presence of tranexamic acid, the lysine receptor binding sites of plasmin for fibrin are occupied, preventing binding to fibrin monomers, thus preserving and stabilizing fibrin's matrix structure.⁶The antifibrinolytic activity of tranexamic acid results in inhibition of the dissolution of clots.³

Clinical studies indicated that a 3900 mg/day regimen (marketed in the US as Lysteda) meets MBL reduction targets established by the FDA and significantly reduces limitations on social, leisure and physical activities.^{5,6}

In the evaluation of tranexamic acid in the treatment of menorrhagia performed in 2000 by the European Agency for the Evaluation of Medicinal Products (EMEA), a dose-dependent increase in efficacy was noted. The same review recommended a daily dose of 3-4 g/day and indicated that the risk of gastrointestinal adverse events is increased at 6 g/day.⁷ While the FDA-approved tranexamic acid regimen is within the aforementioned recommended dosing range, certain Warnings and Precautions—dose adjustment in women with renal impairment; increase in the risk of blood clots, stroke, or myocardial infarction in the event of concomitant therapy with hormonal contraceptives; the possibility of severe allergic reactions; visual or ocular adverse effects⁶—reflect regulatory concerns regarding Lysteda's safety. In the risk-benefit assessment, the FDA medical reviewer suggested a 50% dose reduction for women who do not tolerate the common adverse events associated with the approved treatment regimen.⁵ Taken together, the clinical evidence indicates that the efficacy of oral tranexamic acid in the treatment of menorrhagia must be weighed against the potentially disturbing side effects associated with this medication. When taken via the conventional oral route at approved doses, the drug may raise safety concerns.

In addition to the parenteral drug delivery routes (oral and intravenous), topical administration of tranexamic acid has also been extensively studied. Efficacy of this route was established in a number of clinical studies. ^{8,9,22} In various clinical settings, placebo-adjusted differences in blood loss (indicating a direct drug effect) ranged from 55 mL to 750 mL.^{9,10,11,12} The lower limit of the reported range is above the blood loss decrease (50 mL) considered by the FDA as a desirable menstrual blood loss reduction target.⁵

Local administration of tranexamic acid is proven to be efficacious despite very low, sometimes undetectable, circulating drug levels. For example, use of mouthwash containing tranexamic acid was shown to reduce the incidence of postoperative bleeding complications when compared to placebo.⁸ Such an effect was achieved despite a small amount of tranexamic acid being administered and its short residence time in the oral cavity.³⁰ After administration of an oral tablet of tranexamic acid, mean drug plasma concentrations (7 mcg/mL) reached therapeutic levels, whereas the drug was not detected in saliva samples. After a mouth rinse, plasma concentrations remained below 2 mcg/mL while the saliva concentrations reached a much higher maximum level (above 200 mcg/mL) and remained at a therapeutic level for more than two hours.^{8,13} Reduction of the postoperative blood loss without detectable plasma drug levels was also reported for other locally administered antifibrinolytic drugs, specifically aprotinin.¹⁰

A number of studies have investigated fibrinolytic enzyme systems in menstrual blood.^{14,15,16,17,18,19,20,21} Results of one of these studies strongly suggest that high levels of menstrual plasminogen activator and plasmin are most likely derived from the endometrium, with significantly greater levels in women with excessive bleeding when compared to those with normal menstrual volumes. Notably, no such differences could be found in the peripheral blood. Both plasminogen activator and plasmin activity in menstrual blood were significantly higher than those in peripheral blood irrespective of the intensity of the women's menstrual bleeding.¹⁵

Effectiveness of intravaginal drug delivery is supported by a substantial body of evidence. It is established for estrogens used to treat vaginal atrophy and related symptoms²³, as well as osteoporosis and other menopausal symptoms²⁴. Other examples of compounds efficacious after vaginal administration include (but are not limited to) misoprostol for cervical ripening²⁵, a danazol ring for the treatment of infiltrating endometriosis²⁶, and a progesterone gel.^{27,28} The contraceptive efficacy of the levonorgestrel(LNG)-containing intrauterine system, Mirena® with 20 mcg/day LNG delivery is at least comparable to that reported for the LNG-only pill, delivering a 50% greater daily dose. As noted in the Mirena Medical review, serum concentrations of levonorgestrel for Mirena are approximately one-tenth the serum concentration produced by an oral contraceptive containing 0.1 mg LNG and about half that produced by the Norplant® system. The local endometrial concentrations, however, are over 100 times higher in Mirena users than in users of oral contraceptives containing 0.25 mg LNG.²⁹

### SUMMARY

Certain embodiments disclosed herein provide a method for effectively decreasing menstrual blood loss without the undesirable side effects of current oral medications by providing for intravaginal delivery of an antifibrinolytic or hemostatic agent. More specifically, the present invention relates to the intravaginal delivery of an antifibrinolytic or hemostatic agent using medicated feminine sanitary products (e.g., menstrual pads, such as mini-pads, maxi-pads, sanitary napkins, sanitary towels, sanitary pads, or menstrual tampons). According to the method of the present invention, medicated feminine sanitary products deliver active drug (antifibrinolytic or hemostatic agent) while absorbing menstrual flow (menstrual blood and/or menstrual fluid).

According to one embodiment, medicated feminine sanitary products release an ultra-low dose of tranexamic acid (or another suitable antifibrinolytic or hemostatic agent) directly to the affected tissue(s), particularly the uterine cavity, which is close to the location (the vagina) where the pad or tampon is applied or inserted. As specified herein, effective local concentrations of drug are achievable with doses much lower than those administered intravenously, or by the oral route. When used according to the method of the invention, levels of tranexamic acid (or another antifibrinolytic or hemostatic agent) in the systemic circulation are greatly reduced as compared to oral therapies, possibly below detectable limits, leading to a lower incidence of adverse events, such as diarrhea, nausea, vomiting, allergic reactions, disturbance of color, sharpness, or field of vision, etc. The local administration of tranexamic acid may also eliminate risk of systemic toxicity and thrombloembolism—well known risks associated with its oral and intravenous administration. Relatively high local tissue concentrations of tranexamic acid (or another suitable antifibrinolytic or hemostatic agent) used in the method of the present invention ensure a shorter time to achieving the desired hemostatic changes, including reduction of plasminogen activator and plasmin levels, formation of strong and stable blood clots, etc. Better compliance consistent with the trivial replacement of sanitary products which are already being used during the menstrual period (as opposed to adherence to a pill-taking regimen) is also expected. For a number of drugs delivered intravaginally, the oral route/intravaginal route ratio for the daily doses seems to be close to 10:1. The same ratio may reasonably be assumed for tranexamic acid (or another antifibrinolytic or hemostatic agent).

While the exact intravaginal doses for each drug useful in the method of the present invention are going to be determined in clinical trials, the possibility of a drastic dose decrease, relative to the currently-approved oral doses, with no compromise (but rather improvement) in the reduction of menstrual blood loss is surprising and new. With an expected decrease in drug-related adverse events, this treatment modality may be considered as the first treatment option in the management of heavy menstrual bleeding. If a woman is not satisfied with the ensuing results, other therapeutic interventions (for example, oral tablets at much greater doses) may be considered.

Also, surprising and new is the possibility of achieving a therapeutic effect (manifested in a reduction in menstrual blood loss) in the absence of detectable plasma concentration levels, or in the presence of circulating levels of the drug that is much lower than those reported after administration of oral tablets. Finally, surprising and new is the concept of using routine feminine sanitary products (e.g., menstrual pads or menstrual tampons) as drug delivery devices for the control of menstrual bleeding.

The novelty of this invention is also related to the recommended use of medicated sanitary products for a variety of menstrual conditions, ranging from light discharge to heavy bleeding. Even a moderate menstrual period may have a negative impact on a woman's quality of life, particularly when she experiences days of relatively heavy bleeding and/or prolonged duration of menstrual period. Episodes of the lightest bleeding (“spotting”) may also impact a woman's well-being and her daily routines.

Use of feminine sanitary products medicated with an antifibrinolytic or hemostatic agent may also be a preferred option for women taking hormonal contraceptives and experiencing scheduled and/or unscheduled vaginal bleeding. Women entering the perimenopausal stage with a less predictable menstrual pattern are also expected to benefit from less intense and/or shorter periods. The published findings suggest that plasminogen activator and plasmin activity in menstrual blood are significantly higher than those in peripheral blood irrespective of the intensity of the women's menstrual bleeding.¹⁵. Therefore, use of an antifibrinolytic or hemostatic agent can be beneficial in “normally” menstruating women, giving them greater control over the intensity and duration of menstrual periods.

In addition to tranexamic acid, the reduction of menstrual blood loss may utilize a number of antifibrinolytic and hemostatic agents, including ε-amino-caproic acid, aprotinin, antipan, gabexate mesilate, pepstatin, leupeptin, chymostatin and others. The exact dose of these compounds will be determined in future clinical trials. Initial dose selection will be driven by a number of factors, including but not limited to the potency of the compound tested, the amount of menstrual flow and the presence of menstrual blood clots and stains in patients who need treatment. The severity of the symptoms associated with menstruation, as well as patient characteristics (age, weight, presence of anemia, duration of the disease, etc.).

### DETAILED DESCRIPTION

The embodiments disclosed herein are only examples of the many possible advantageous uses and implementations of the innovative teachings presented herein. In general, statements made in the specification of the present application do not necessarily limit any of the various claimed embodiments. Moreover, some statements may apply to some inventive features but not to others.

### Definitions:

A feminine sanitary product is defined as an absorbent item worn by a woman during the menstrual period (for example, a menstrual pad, a maxi-pad, a mini-pad, a sanitary napkin, a sanitary towel, a sanitary pad, or a menstrual tampon).

Menstrual flow is defined as encompassing menstrual blood and/or menstrual fluid.

A therapeutically effective amount of an antifibrinolytic or hemostatic agent is defined as the amount of a drug that results in a significant (preferably, at least 15%) reduction in menstrual blood loss when compared to the pre-treatment levels.

Certain disclosed embodiments provide for intravaginal delivery of a therapeutically effective amount of an antifibrinolytic or hemostatic agent for the reduction of menstrual blood loss and an improvement in related symptoms.

In a preferred embodiment, the antifibrinolytic or hemostatic agent is delivered on a feminine sanitary product. In one embodiment, the antifibrinolytic or hemostatic agent is administered from the onset of menstrual bleeding until the resolution of related symptoms or the end of the menstrual period. In another embodiment, the feminine sanitary product is selected from the group consisting of a menstrual pad, a maxi-pad, a mini-pad, a sanitary napkin, a sanitary towel, a sanitary pad, and a menstrual tampon.

The drug formulation of embodiments of the disclosed technology is administered to females desiring to reduce menstrual blood loss at the time of menstrual bleeding, irrespective of the specific volume of menstrual blood loss (ranging from light discharge to extremely heavy bleeding).

In a preferred embodiment, the drug delivery device useful in the methods of the present invention is a feminine sanitary product (e.g., a menstrual pad, a maxi-pad, a mini-pad, a sanitary napkin, a sanitary towel, a sanitary pad, or a menstrual tampon) with a design known in the art, which is designed to deliver active drug while absorbing menstrual flow (menstrual blood and/or menstrual fluid). In one embodiment, the active drug can be mixed throughout the feminine sanitary product. In another embodiment, the active drug can be distributed uniformly throughout the feminine sanitary product. In yet another embodiment, the active drug can be located in a part of the feminine sanitary product. In a further embodiment, the active drug can be located in a part of the feminine sanitary product selected from the center, one side, or outer surface attached to an inner layer (core).

The feminine sanitary product (e.g., a menstrual pad, a maxi-pad, a mini-pad, a sanitary napkin, a sanitary towel, a sanitary pad, or a menstrual tampon) can utilize any other design providing absorption of menstrual flow (menstrual blood and/or menstrual fluid) along with drug release to the local affected tissues (e.g., uterine cavity).

In certain embodiments, the active drug can be incorporated into the feminine sanitary product as a pharmaceutically acceptable composition selected from the group consisting of a liquid solution, a capsule, a membrane, a drug-releasing strip, a tablet, powder, and gel. In another embodiment, the feminine sanitary product (e.g., a menstrual pad, a maxi-pad, a mini-pad, a sanitary napkin, a sanitary towel, a sanitary pad, or a menstrual tampon) can be designed as a fluid-expandable menstrual tampon. In yet another embodiment, the feminine sanitary product can be designed as a menstrual pad (or sanitary napkin, or sanitary towel, or sanitary pad) with a protuberance projecting beyond the vaginal opening (introitus, vaginal orifice) into the vagina. In a further embodiment, the feminine sanitary product can be designed as a menstrual pad (or sanitary napkin, or sanitary towel, or sanitary pad) having a segment or region facing the vagina. In another embodiment, the feminine sanitary product can be designed as a menstrual pad (or sanitary napkin, or sanitary towel, or sanitary pad) penetrating into the vagina. In yet another embodiment, the feminine sanitary product can be designed as a menstrual pad (or sanitary napkin, or sanitary towel, or sanitary pad) with contact between a part of the pad and the vaginal walls.

In certain embodiments, the medicated menstrual tampon is inserted in the vagina in the same fashion as recommended for respective feminine sanitary products (menstrual tampons). In another embodiment, the medicated menstrual pad (or sanitary napkin, or sanitary towel, or sanitary pad) is utilized in the same fashion and regimen as recommended for respective feminine sanitary products, e.g., menstrual pads, sanitary napkins, sanitary towels, or sanitary pads.

The use of feminine sanitary products (e.g., a menstrual pad, a maxi-pad, a mini-pad, a sanitary napkin, a sanitary towel, a sanitary pad, or a menstrual tampon) delivering drug directly to the affected tissues is expected to enhance the drug's efficacy in the reduction of the volume of menstrual blood loss and improvement in the related symptoms and a woman's quality of life; it may also result in a shorter duration of menstrual bleeding. The use of feminine sanitary products (e.g., a menstrual pad, a maxi-pad, a mini-pad, a sanitary napkin, a sanitary towel, a sanitary pad, or a menstrual tampon) delivering drug directly to the affected tissues is also expected to significantly reduce the daily dose when compared to other routes of drug administration; this may result in a lower systemic drug circulation, possibly below detectable levels, and a lower incidence of drug-related adverse events.

In all embodiments, the active ingredient is from a class of drugs called antifibrinolytic agents or the hemostatic agents, including tranexamic acid, ε-amino-caproic acid, aprotinin, antipan, gabexate mesilate, pepstatin, leupeptin, chymostatin and others, or from a class of drugs called hemostatic agents and metabolites thereof. Also in all embodiments, a feminine sanitary product (e.g., a menstrual pad, a maxi-pad, a mini-pad, a sanitary napkin, a sanitary towel, a sanitary pad, or a menstrual tampon) is medicated with an amount of active drug ranging from 50 mg to 1 g.

In a preferred embodiment, a feminine sanitary product (e.g., a menstrual pad, a maxi-pad, a mini-pad, a sanitary napkin, a sanitary towel, a sanitary pad, or a menstrual tampon) is medicated with an amount of active drug ranging from 100 mg to 500 mg. In another embodiment, a feminine sanitary product (e.g., a menstrual pad, a maxi-pad, a mini-pad, a sanitary napkin, a sanitary towel, a sanitary pad, or a menstrual tampon) is medicated with amount of active drug ranging from 100 mg to 300 mg. In yet another embodiment, the active ingredient is tranexamic acid with the amount of active drug per medicated sanitary product ranging from 100 mg to 300 mg. In a further embodiment, the active ingredient is ε-amino-caproic acid with the amount of active drug per medicated sanitary product ranging from 200 mg to 400 mg. In another preferred embodiment, the active ingredient is aprotinin with the amount of active drug per medicated sanitary product ranging from 150 mg to 300 mg.

In certain embodiments, the drug formulation treats females with menstrual bleeding of less than 80 ml per menstrual cycle. In other embodiments, the drug formulation treats females with menstrual bleeding of more than 80 ml per menstrual cycle. In yet other embodiments, the drug formulation treats females clinically diagnosed with menorrhagia. In further embodiments, the drug formulation treats females clinically diagnosed with idiopathic menorrhagia. In other embodiments, the drug formulation treats females clinically diagnosed with cyclic heavy menstrual bleeding. In yet other embodiments, the drug formulation treats females clinically diagnosed with dysfunctional uterine bleeding. In further embodiments, the drug formulation treats females with no clinical diagnosis related to menorrhagia, idiopathic menorrhagia, cyclic heavy menstrual bleeding, or dysfunctional uterine bleeding, but who perceive their menstrual periods to be heavy. In other embodiments, the drug formulation treats females clinically diagnosed with anemia.

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLE 1

The feminine sanitary product serving as a drug delivery device is a menstrual tampon comprised of a conventional menstrual tampon base with pharmaceutical dosage component(s) attached to it. The latter are represented by 2-3 capsules. Each capsule contains a 10-15% aqueous solution of tranexamic acid. The total amount of drug per tampon ranges from 100 to 150 mg. Alternatively, pharmaceutically acceptable solution of ε-amino-caproic acid is used with the total amount of drug ranging from 200 mg to 400 mg. Alternatively, pharmaceutically acceptable solution of aprotinin is used with the total amount of drug ranging from 150 mg to 200 mg. Capsules are dissolved by the body's heat after insertion of the tampon. The material from which the capsule is constructed must be easily rupturable. Examples of such materials include gelatin and various synthetic resins known in the art.

The capsules are located on the entering end of tampon opposite to the conventional inserter device (cartridge) and/or withdrawal string of the tampon. Alternatively, they are located on opposite sides along the body of the tampon. The capsules have a mushroom shape with a stem portion at least partially embedded in the body of the tampon. The embedded portion is made from fluid-impermeable material. Alternatively, the capsules are placed on a surface of the body of the tampon and separated from that body by the patches made from fluid-impermeable material.

This embodiment is expected to control moderate-to-heavy menstrual bleeding. Method and frequency of use of the described device are the same as routinely employed for non-medicated menstrual tampons.

### EXAMPLE 2

The feminine sanitary product serving as a drug delivery device is a menstrual tampon comprising of a conventional menstrual tampon base with pharmaceutical dosage component attached to it. The latter are represented by a vaginal tablet containing 100 to 150 mg of tranexamic acid. Alternatively, a vaginal tablet contains 200 mg to 300 mg of ε-amino-caproic acid. Alternatively, a vaginal tablet contains 150 mg to 200 mg of aprotinin. The tablet is slowly melted by the body's heat after insertion of the tampon.

The tablet is located on the entering end of tampon opposite to the conventional inserter device (cartridge) and/or withdrawal string of the tampon. The tablet is placed on a “base” partially embedded in the body of the tampon. The embedded portion is made from fluid-impermeable material. Alternatively, the tablet is placed on a surface of the body of the tampon and separated from that body by a patch made from fluid-impermeable material.

This embodiment is expected to control moderate-to-heavy menstrual bleeding. Method and frequency of use of the described device are the same as routinely employed for non-medicated menstrual tampons.

### EXAMPLE 3

The feminine sanitary product serving as a drug delivery device is a menstrual tampon with a resilient foam corpus overwrapped with a medicament-impregnated covering. Detailed description of such tampon may be found in U.S. Pat. No. 3,902,493.

As is explained above, to ensure delivery of medications to the afflicted areas of the vagina, the suitable foams have a modulus of compression which allows the tampon to be reformed following insertion to conform to the irregularities of the vaginal wall and to achieve adequate vaginal contact (foam expandable by menstrual fluids is particularly suitable.)

In addition to the foam corpus, the tampon has a medicament-bearing hydrophobic non-woven overwrap permeable to vaginal fluids.

Per U.S. Pat. No. 3,902,493, the medicament composition is applied to a portion of the overwrap surface, thereby assuring that the absorptive properties of the tampon are not hindered and that the tampon performs adequately when worn during menstruation. The medicament composition represents an appropriate formulation of tranexamic acid. It is in the form of binding lubricant (as described in the referenced patent). Alternatively, it is in a form of 10% aqueous solution (100 mg of tranexamic acid in 1 mL of water), or a concentrated 25% solution (250 mg of tranexamic acid in 1 mL of water). Gel formulations of tranexamic acid (known in the art) may also be considered. The total amount of tranexamic acid per tampon ranges from 150 to 200 mg.

Alternatively, pharmaceutically acceptable solution of ε-amino-caproic acid is used with the total amount of drug ranging from 250 mg to 400 mg. Alternatively, pharmaceutically acceptable solution of aprotinin is used with the total amount of drug ranging from 200 mg to 250 mg.

Use of this embodiment is control of heavy menstrual bleeding. Method and frequency of use of the described device are the same as routinely employed for non-medicated menstrual tampons.

### EXAMPLE 4

The feminine sanitary product serving as a drug delivery device is a menstrual tampon in combination with drug-containing reservoir. Detailed description of such tampon may be found in one of the embodiments of U.S. Pat. No. 4,318,405 which is herein incorporated by reference.

As is explained in the patent description, an important feature is the design of the tampon inserter permitting pre-wetting of the tampon prior to insertion into the vaginal cavity. In the referenced patent, the tampon and drug delivery device are submerged in a wetting solution, such as water, immediately prior to insertion. As a modification of this approach, the tampon inserter contains a 15% aqueous solution of tranexamic acid, with approximately 150 mg of drug available for delivery into the vaginal cavity. Alternatively, pharmaceutically acceptable solution of ε-amino-caproic acid is used with the total amount of drug ranging from 250 mg to 400 mg. Alternatively, pharmaceutically acceptable solution of aprotinin is used with the total amount of drug ranging from 150 mg to 200 mg. Pre-wetting creates a tampon impregnated with a therapeutically effective amount of tranexamic acid.

Use of this embodiment is control of moderate-to-heavy menstrual bleeding. Method and frequency of use of the described device are the same as routinely employed for non-medicated menstrual tampons.

### EXAMPLE 5

The feminine sanitary product serving as a drug delivery device is a menstrual tampon adapted for carrying a medicament for selective expulsion during use. Detailed description of such tampon may be found in the U.S. Pat. No. 5,273,521 which is herein incorporated by reference. As it is explained in the patent description, a tampon has a tubular inserter, closed on one end, on which an elongated tampon body is mounted. A medicament is disposed in a longitudinal bore extending through the tampon body. A specially designed wand moves the medicament composition out of the bore into the vaginal cavity. After the medication is completely expelled, the wand is moved in the opposite direction until it is further withdrawn from the tampon body and, subsequently, from the vaginal cavity. (Another attractive option is to keep the bore within the tampon to provide continuous drug release into the vaginal cavity). The tampon's body is being comprised of an absorbent material having porous outer and inner surfaces. The string attached to the body of the tampon is necessary to remove the tampon after use.

The medicament composition represents an appropriate formulation of tranexamic acid. It is in form of 10% aqueous solution (100 mg of tranexamic acid in 1 mL of water), or concentrated 25% solution (250 mg of tranexamic acid in 1 mL of water). Gel formulations of tranexamic acid (known in the art) may also be considered. Alternatively, pharmaceutically acceptable solution of ε-amino-caproic acid is used with the total amount of drug ranging from 250 mg to 400 mg. Alternatively, pharmaceutically acceptable solution of aprotinin is used with the total amount of drug ranging from 200 mg to 250 mg.

Use of this embodiment is control of moderate-to-heavy menstrual bleeding. Method and frequency of use of the described device are the same as routinely employed for non-medicated menstrual tampons.

### EXAMPLE 6

The feminine sanitary product serving as a drug delivery device is a menstrual tampon in combination with a drug delivery device attached to its body. The drug delivery device is represented by 2-4 strips positioned alongside the body of the tampon. Alternatively, these strips are positioned around the width of the body of the tampon. The strips are made from polymers known in the art (for example, polyethylene) and separated from the body of the tampon by liquid-impermeable material. An aqueous solution of tranexamic acid or any other appropriate formulation (such as an acetic acid-based solution of tranexamic acid, an ethanolic solution of tranexamic acid, or tranexamic acid powder) is deposited on the strips using manufacturing methods known in the art. Appropriate wetting agents, surfactants and excipients are added as necessary. The total amount of tranexamic acid per tampon ranges from 100 to 150 mg. Alternatively pharmaceutically acceptable solution of ε-amino-caproic acid is used with the total amount of drug ranging from 200 mg to 350 mg. Alternatively, pharmaceutically acceptable solution of aprotinin is used with the total amount of drug ranging from 150 mg to 200 mg.

Use of this embodiment is control of moderate-to-heavy menstrual bleeding. Method and frequency of use of the described device are the same as routinely employed for non-medicated menstrual tampons.

### EXAMPLE 7

The drug delivery device is a menstrual pad with a protuberance designed to project beyond the vaginal opening (introitus, vaginal orifice) into the lower vagina. The protuberance represents a multi-dimensional, elongated body of fluid-impermeable material attached to the skin-facing region of the pad. It is approximately 2-2.5 inches long and 1-1.5 inches wide. The rest of the body of the pad is planar and elongated; it resembles the design of commercially available menstrual pads such as Kotex Maximum Protection or Always Maxi pads.

The protuberance, on its vaginal-facing aspect, is encapsulated within one or more rupturable membranes or capsules. Membranes or capsules containing tranexamic acid are ruptured as a result of a slight mechanical pressure when a menstrual pad is attached to the body (and the protuberance is projected into the vagina). Alternatively, they are dissolved by the body's heat. The membranes or capsules contain a 10% aqueous solution of tranexamic acid, with the total amount of drug being approximately 200 mg dilluted in 2 mL of water. Alternatively, pharmaceutically acceptable solution of ε-amino-caproic acid is used with the total amount of drug ranging from 250 mg to 400 mg. Alternatively, pharmaceutically acceptable solution of aprotinin is used with the total amount of drug ranging from 250 mg to 300 mg. The material of membrane or capsule must be easily rupturable. Examples of such materials include gelatin and various synthetic resins known in the art.

Use of this embodiment is control of light-to-moderate menstrual bleeding and occasional spotting. Method and frequency of use of the described device are the same as routinely employed for non-medicated menstrual pads.

### EXAMPLE 8

The drug delivery device is a planar and elongated menstrual pad; it resembles the design of commercially available menstrual pads such as Kotex® Maximum Protection or Always® Maxi pads.

The pad, on its vaginal-facing aspect, is encapsulated within one or more rupturable membranes or capsules. Membranes or capsules containing tranexamic acid are ruptured as a result of a slight mechanical pressure when a menstrual pad is attached to the body. Alternatively, they are dissolved by the body's heat. The membranes or capsules contain a 10% aqueous solution of tranexamic acid, with the total amount of drug being approximately 200 mg diluted in 2 mL of water. Alternatively, pharmaceutically acceptable solution of ε-amino-caproic acid is used with the total amount of drug ranging from 250 mg to 400 mg. Alternatively, pharmaceutically acceptable solution of aprotinin is used with the total amount of drug ranging from 250 mg to 300 mg. The material of membrane or capsule must be easily rupturable. Examples of such materials include gelatin and various synthetic resins known in the art.

Use of this embodiment is control of light-to-moderate menstrual bleeding and occasional spotting. Method and frequency of use of the described device are the same as routinely employed for non-medicated menstrual pads.

### EXAMPLE 9

The drug delivery device is a menstrual pad with a protuberance designed to project beyond vaginal opening (introitus, vaginal orifice). The protuberance represents a multi-dimensional, elongated body of fluid-impermeable material attached to the skin-facing region of the pad. It is approximately 2-2.5 inches long and 1-1.5 inches wide. The rest of the body of the pad is planar and elongated; it resembles the design of commercially available menstrual pads such as Kotex Maximum Protection or Always Maxis pads.

The protuberance, on its vaginal-facing aspect, contains 1-3 strips positioned alongside the body of the protuberance. Alternatively, these strips are positioned around the width of the body of the protuberance. The strips are made from polymers known in the art (for example, polyethylene) and separated from the body of the pad by liquid-impermeable material. Tranexamic acid aqueous solution or any other appropriate formulation (such as an acetic acid-based solution or an ethanolic solution of tranexamic acid) is deposited on the strips. Appropriate wetting agents or surfactants are added as necessary. The total amount of tranexamic acid per pad ranges from 100 to 150 mg. Alternatively, pharmaceutically acceptable solution of ε-amino-caproic acid is used with the total amount of drug ranging from 200 mg to 300 mg. Alternatively, pharmaceutically acceptable solution of aprotinin is used with the total amount of drug ranging from 200 mg to 250 mg.

Use of this embodiment is control of moderate-to-heavy menstrual bleeding. The method and frequency of use of the described device are the same as routinely employed for non-medicated menstrual pads.

### EXAMPLE 10

The drug delivery device is a planar and elongated menstrual pad without a protuberance; it resembles the design of commercially available menstrual pads such as Kotex Maximum Protection or Always Maxis pads.

The pad, on its vaginal-facing aspect, contains 1-3 strips positioned alongside the body of the pad. The strips are made from polymers known in the art (for example, polyethylene) and separated from the body of the tampon by liquid-impermeable material. An aqueous solution of tranexamic acid, or any other appropriate formulation (such as an acetic acid-based solution or an ethanolic solution of tranexamic acid) is deposited on the strips. Appropriate wetting agents or surfactants are added as necessary. The total amount of tranexamic acid per pad ranges from 100 to 150 mg. Alternatively, pharmaceutically acceptable solution of ε-amino-caproic acid is used with the total amount of drug ranging from 200 mg to 300 mg. Alternatively, pharmaceutically acceptable solution of aprotinin is used with the total amount of drug ranging from 200 mg to 250 mg.

Use of this embodiment is control of moderate-to-heavy menstrual bleeding. Method and frequency of use of the described device are the same as routinely employed for non-medicated menstrual pads.

### EXAMPLE 11

The drug delivery device is a combination of a tampon and a menstrual pad. The possible design of such combination is described in the U.S. Pat. No. 6,059,763 which is herein incorporated by reference. The combination consists of a base pad having first and second length sides and first and second wings. The combination also includes a tampon extending substantially perpendicular to the base pad at its central portion. The tampon is positioned within the body opening.

The referenced patent describes a non-medicated feminine sanitary product. With the objective of the present invention in mind—intravaginal administration of tranexamic acid to control menstrual bleeding—the non-medicated tampon is replaced with the devices described earlier. Particularly, the use of tampons as described in detail above is recommended. See related descriptions above for further information regarding tampon design, the dose of tranexamic acid and the use of the drug delivery device(s).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

¹ Heavy menstrual bleeding: Assessing Impact, Evaluating Management Options; Supplement to OBG Management, October 2009; accessed at http://www.obgmanagement.com/PDF/supplOBG heaveybleeding.pdf (reference on file)
² Andrew M. Kaunitz Modern Management of Heavy Menstrual Bleeding (reference on file)
³ Joseph Y. Lee, et al. Treatment of Menorrhagia with Tranexamic Acid. J Soc Obstet Gynaecol Can 2000;22(10):794-8.
⁴ National Collaborating Centre for Women's and Children's Health. Heavy menstrual bleeding. London (UK): Royal College of Obstetricians and Gynaecologists (RCOG); 2007 January.
⁵ Center for Drug Evaluation and Research. Application Number 22-430. Lysteda® NDA Medical review; accessed at http://www.accessdata,fda.gov/drugsatfda docs/nda/2009/022430s000medr.pdf (reference on file)
⁶Lysteda®. Full Prescribing Information; accessed at http://www.lysteda.com/assets/pdf/PI FERRING2010.pdf (reference on file)
⁷ Overall Summary of the Scientific Evaluation of Cycle-f, EMEA, 2000.
⁸ Dunn C J, Goa K L. Tranexamic acid: a review of its use in surgery and other indications. Drugs. 1999;57:1005-32.
⁹Jabalameli M., Zakeri K. Evaluation of Topical Tranexamic Acid on Intraoperative Bleeding in Endoscopic Sinus Surgery Iran J Med Sci December 2006; Vol 31 No 4 221
¹⁰De Bonnis M. et al. Topical use of tranexamic acid in coronary artery bypass operations: a double-blind, prospective, randomized, placebo-controlled study. J Thorac Cardiovasc Surg. 2000 March;119(3):575-80
¹¹Fawzy H, et al. Can local application of Tranexamic acid reduce post-coronary bypass surgery blood loss? A randomized controlled trial. J Cardiothorac Surg. 2009 June 18;4:25
¹²Vlessides M. Topical Tranexamic Acid Cuts Bleeding After Knee Surgery. Clinical Anesthesiology. February 2010|VOLUME: 36:2
¹³Sindet-Pedersen S. Distribution of tranexamic acid to plasma and saliva after oral administration and mouth rinsing: a pharmacokinetic study. J Clin Pharmacol 1987;27:1005-8.
¹⁴Gleeson N C et al. The effect of tranexamic acid on measured menstrual loss and endometrial fibrinolytic enzymes in dysfunctional uterine bleeding. Acta Obstetricia et Gynecologica Scandinavica 1994 73:3, 274-277
¹⁵Dockeray C J. et al. The fibrinolytic enzyme system in normal menstruation and excessive uterine bleeding and the effect of tranexamic acid. Eur J Obstet Gynecol Reprod Biol. 1987 April;24(4):309-18
¹⁶Rybo G. Plasminogen activators in endometrium. Acta Obstet Gynecol Scand. 1966; 45:411-449
¹⁷Hefnawi A S. et al. Fibrinolytic activity of menstrual blood in normal and menorrhagic women and in women wearing the Lippes Loop and Cu-T (200). Int J Gynaecol Obstet 1979; 16:400-407.
¹⁸Hahn L. et al. Blood coagulation, fibrinolysis and plasma protein in women with normal and with excessive menstrual blood loss. Br J Obstet Gynaecol 1976; 83: 974-980.
¹⁹Cole S K. Clarkson A R. Menstrual blood loss and fibrin degradation products. Br Med J 1972; 1:78-79.
²⁰Rees M C P et al. Coagulation factors and fibrinolytic proteins in menstrual fluid collected from normal and menorrhagic women. Br J Obstet Gynaecol 1985; 92: 1164-1168.
²¹Basu H K. Fibrin degradation products in sera of women with normal menstruation and menorrhagia. Br Med J 1970; 1:74-75.
²² Boethius J. “Method and hemostatic patch for effecting local hemostasis”, U.S. Pat. No. 7,022,125, Issued on Apr. 4, 2006.
²³Kingsberg S A., et al. Treating dyspareunia caused by vaginal atrophy: a review of treatment options using vaginal estrogen therapy. International Journal of Women's Health. August 2009, Volume 2009:1 Pages 105-111
²⁴Ballagh S A. Vaginal Ring Hormone Delivery Systems in Contraception and Menopause. Clinical obstetrics and gynecology/volume 44/number 1/Mar. 2001
²⁵Iyer V., et al. Vaginal drug delivery. ExpressPharma, 1-15 Jul. 2008
²⁶Igarashi M. Novel vaginal danazol ring therapy for pelvic endometriosis, in particular deeply infiltrating endometriosis. Hum Reprod. 1998 July;13(7):1952-6.
²⁷Ficicioglu C. et al. High local endometrial effect of vaginal progesterone gel. Gynecological Endocrinology, Volume 18, Issue 5 May 2004, pages 240-243.
²⁸Kuhl H. Pharmacology of estrogens and progestogens: influence of different routes of administration. Climacteric 2005; 8(Suppl 1)3-63.
²⁹ Center for Drug Evaluation and Research. Application Number 21-225. Mirena® NDA Medical review; accessed at Mirena® NDA Medical review; accessed at http://www.accessdata.fda.gov/drugsatfda docs/nda/2000/21-225.pdf Mirena Medr.pdf (reference on file)
³⁰ Randall C. U.K. National Health Service. Surgical management of the primary care dental patient on warfarin. March 2007. Accessed at hitp://www.dundee.ac.uk/tuith/Static/info/warfarin.pdf (reference on file)

What is claimed is:

1. A method for reducing menstrual blood loss in a female, comprising:
delivering an active agent to the uterine cavity by administering intravaginally to the female a therapeutically effective amount of the active agent on a medicated feminine sanitary product during menstrual bleeding, wherein the medicated feminine sanitary product is routinely utilized to absorb the menstrual flow, wherein the active agent is incorporated in the medicated feminine sanitary product as a pharmaceutically acceptable composition, the active agent is any of an antifibrinolytic and a hemostatic agent.

2. The method of claim 1, wherein the feminine sanitary product is any one of: a menstrual pad, a maxi-pad, a mini-pad, a sanitary napkin, a sanitary towel, a sanitary pad, and a menstrual tampon.

3. The method of claim 1, wherein the pharmaceutically acceptable composition includes at least one of: a liquid solution, a capsule, a membrane, a drug-releasing strip, a tablet, powder, and gel.

4. The method of claim 1, wherein the any one of the antifibrinolytic and the hemostatic agent comprises any one of: tranexamic acid, ε-amino-caproic acid, aprotinin, antipan, gabexate mesilate, pepstatin, leupeptin, chymostatin, and metabolites thereof.

5. The method of claim 4, wherein the feminine sanitary product comprises an amount of the active agent ranging from 50 mg to 1 g.

6. The method of claim 4, wherein the feminine sanitary product comprises an amount of the active agent ranging from 100 mg to 500 mg.

7. The method of claim 6, wherein the active agent is tranexamic acid and the amount of the tranexamic acid in the feminine sanitary product ranges from 100 mg to 300 mg.

8. The method of claim 6, wherein the active agent is ε-amino-caproic acid and the amount of the ε-amino-caproic acid in the feminine sanitary product ranges from 200 mg to 400 mg.

9. The method of claim 6, wherein the active agent is aprotinin and the amount of the aprotinin in the feminine sanitary product ranges from 150 mg to 300 mg.

10. The method of claim 1, wherein the female suffers from a medical condition comprising any one of: menorrhagia, idiopathic menorrhagia, cyclic heavy menstrual bleeding, dysfunctional uterine bleeding, and anemia.

11. A medicated feminine sanitary product, comprising:
a therapeutically effective amount of an active agent that reduces menstrual blood loss, the active agent being delivered to the uterine cavity, wherein the medicated feminine sanitary product is routinely utilized to absorb the menstrual flow, wherein the active agent is incorporated in the medicated feminine sanitary product as a pharmaceutically acceptable composition, the active agent is any one of an antifibrinolytic and a hemostatic agent.

12. The medicated feminine sanitary product of claim 11, wherein the feminine sanitary product is any one of: a menstrual pad, a maxi-pad, a mini-pad, a sanitary napkin, a sanitary towel, a sanitary pad, and a menstrual tampon.

13. The medicated feminine sanitary product of claim 12, wherein the active agent contained in the menstrual tampon in at least one of the following forms: uniformly distributed within part of the menstrual tampon, and located in a part of the menstrual tampon selected from a center, one side, or outer surface attached to the inner layer of the menstrual tampon.

14. The medicated feminine sanitary product of claim 12, wherein the menstrual pad is designed to include a protuberance facing the vagina or a segment facing the vagina, wherein the active agent is incorporated in the menstrual pad as a pharmaceutically acceptable composition.

15. The medicated feminine sanitary product of claim 11, wherein the pharmaceutically acceptable composition comprises any one of: a liquid solution, a capsule, a membrane, a drug-releasing strip, a tablet, powder, and gel.

16. The medicated feminine sanitary product of claim 11, wherein the any one of the antifibrinolytic and the hemostatic agent comprises any one of: tranexamic acid, ε-amino-caproic acid, aprotinin, antipan, gabexate mesilate, pepstatin, leupeptin, chymostatin, and metabolites thereof.

17. The medicated feminine sanitary product of claim 16, wherein the medicated feminine sanitary product includes an amount of the active agent ranging from 50 mg to 1 g.

18. The medicated feminine sanitary product of claim 16, wherein the medicated feminine sanitary product includes an amount of the active agent ranging from 100 mg to 500 mg.

19. The medicated feminine sanitary product of claim 18, wherein the active agent is tranexamic acid and the amount of the tranexamic acid in the medicated feminine sanitary product ranges from 100 mg to 300 mg.

20. The medicated feminine sanitary product of claim 18, wherein the active agent is ε-amino-caproic acid and the amount of the ε-amino-caproic acid in the medicated feminine sanitary product ranges from 200 mg to 400 mg.

21. The medicated feminine sanitary product of claim 18, wherein the active agent is aprotinin and the amount of the aprotinin in the medicated feminine sanitary product ranges from 150 mg to 300 mg.

22. A pharmaceutical composition for intravaginal administration, comprising:
a therapeutically effective amount of an active agent that reduces menstrual blood loss, wherein the active agent is any one of an antifibrinolytic and a hemostatic agent, wherein the composition is being delivered on a feminine sanitary product.

23. The composition of claim 22, wherein the composition is formulated as any one of: an aqueous and a pharmaceutically acceptable solution of the active agent, wherein the formulation is in a form comprising any one of: liquid, foam, cream, ointment, and gel.

24. The composition of claim 22, wherein the composition is formulated as a tablet.

25. The composition of claim 22, wherein the feminine sanitary product comprises any one of: a menstrual pad, a maxi-pad, a mini-pad, a sanitary napkin, a sanitary towel, a sanitary pad, and a menstrual tampon.

26. The composition of claim 22, wherein the any of the antifibrinolytic and the hemostatic agent comprises any of: tranexamic acid, ε-amino-caproic acid, aprotinin, antipan, gabexate mesilate, pepstatin, leupeptin, chymostatin, and metabolites thereof.

27. The composition of claim 22, wherein the composition is delivered on the feminine sanitary product in an amount of the active agent ranging from 50 mg to 1 g.

28. The composition of claim 22, wherein the composition is delivered on the feminine sanitary product in an amount of the active agent ranging from 100 mg to 500 mg.

29. The composition of claim 28, wherein the active agent comprises any one of:
tranexamic acid and the amount of the tranexamic acid contained in the feminine sanitary product ranges from 100 mg to 300 mg, ε-amino-caproic acid and the amount of the ε-amino-caproic acid contained in the feminine sanitary product ranges from 200 mg to 400 mg, and aprotinin and the amount of the aprotinin contained in the feminine sanitary product ranges from 150 mg to 300 mg.
